(19) United States
(12) Patent Application Publication
(10) Pub. No.: US 2010/0312241 A1
(43) Pub. Date: Dec. 9, 2010

(54) IMPLEMENTATION OF LIGHT SOURCES WITH ELECTOCAUTERY UNITS

(76) Inventor: Jerry Martin Erickson, JR., Conway, AR (US)

Correspondence Address: Jerry Martin Erickson Jr., 29 Odom Rd., Conway, AZ, 72032, US

(21) Appl. No.: 12/769,857

(22) Filed: Apr. 29, 2010

Related U.S. Application Data
(60) Provisional application No. 61/185,499, filed on Jun. 9, 2009.

(51) Int. Cl. A61B 18/14

(52) U.S. Cl. 606/49

(57) ABSTRACT

An electrocautery unit includes a light source that may be molded to the electrocautery tip during the manufacturing process. The light source may be chosen by the manufacturer that will work with the electrical system used to power the electrocautery. The light source may be monopolor (on demand lighting when the user initiates the electrocautery), bipolar with constant illumination, or separately wired from the electrocautery device and using its own power supply and on and off switch. The shape and/or angle of the light source may be determined by the function desired by the user. A broad angled light source could be used for large areas needing homeostasis. A narrow light could be used to direct illumination directly ahead of the electrocautery unit. The light source may be a light emitting diode (LED), as such a light source may be reusable through gas sterilization or a single use disposable item.

### CROSS-REFERENCE TO RELATED APPLICATIONS

The present application claims benefit of U.S. Provisional Patent application No. 61/185,499, filed Jun. 9, 2009, herein incorporated by reference.

### BACKGROUND OF THE INVENTION

The present invention relates to electrocautery units and, more particularly, to the implementation of light sources with electrocautery units.

Electrocautery is a surgical technique which involves introducing high frequency current to a specific area of the body in order to remove unwanted tissue, seal off blood vessels, or to create a surgical incision. An electrocautery unit uses a very high frequency, usually upwards of 100 kHz, to ensure that the patient's nerves and muscles are not stimulated. Lower frequencies could cause twitching and cramps, which would be a serious problem. Depending on the voltage used, the electrocautery can have varying effects on the patient's body. Electrocautery can be used to cut through soft tissue to access a surgical site, and it can also be used to seal off bleeding blood vessels during surgery to keep the site clean and reduce blood loss. Recovery from surgeries where electrocautery was used can also be more rapid than recovery from conventional surgeries, and the risk of infection can be reduced.

However, there are some cautions involved when using electrocautery. It is important to use the equipment properly and to keep it in good working order to ensure that only the area of interest is coming into contact with heat or electrical current. If electrocautery is not performed with safe equipment, the patient's body could potentially be burned elsewhere.

As can be seen, there is a need for an electrocautery unit having an integral light source allowing the user to accurately perform electrocautery.

### SUMMARY OF THE INVENTION

In one aspect of the present invention, an electrocautery device comprises an electrocautery tip; and a light source to supply light to at least the electrocautery tip.

In another aspect of the present invention, an electrocautery device comprises an electrocautery tip; and a light emitting diode adapted to supply light to at least the electrocautery tip, wherein the light emitting diode is molded into the electrocautery tip.

These and other features, aspects and advantages of the present invention will become better understood with reference to the following drawings, description and claims.

### BRIEF DESCRIPTION OF THE DRAWINGS

FIG. 1 is a cross-sectional view of an electrocautery tip according to an embodiment of the present invention; and

FIG. 2 is a cross-sectional view of an electrocautery tip according to a further embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides an electrocautery tip that includes a light source. The light source may be molded to the electrocautery tip or handle during the manufacturing process. The light source may be chosen by the manufacturer that will work with the electrical system used to power the electrocautery. The light source may be monopolor (on demand lighting when the user initiates the electrocautery), bipolar with constant illumination, or separately wired from the electrocautery device and using its own power supply and on and off switch. The on and off switch may be, for example, a floor pedal switch. The shape and/or angle of the light source may be determined by the function desired by the user. A broad angled light source could be used for large areas needing homeostasis. A narrow light could be used to direct illumination directly ahead of the electrocautery unit. The light source may be a light emitting diode (LED), as such a light source may be reusable through gas sterilization.

Currently, there must be at least two or three systems used to ensure good electrocautery. There must be an electrocautery unit, room lights, and/or a headlight worn by the medical professional to see inside of a surgical site that is being operated on.

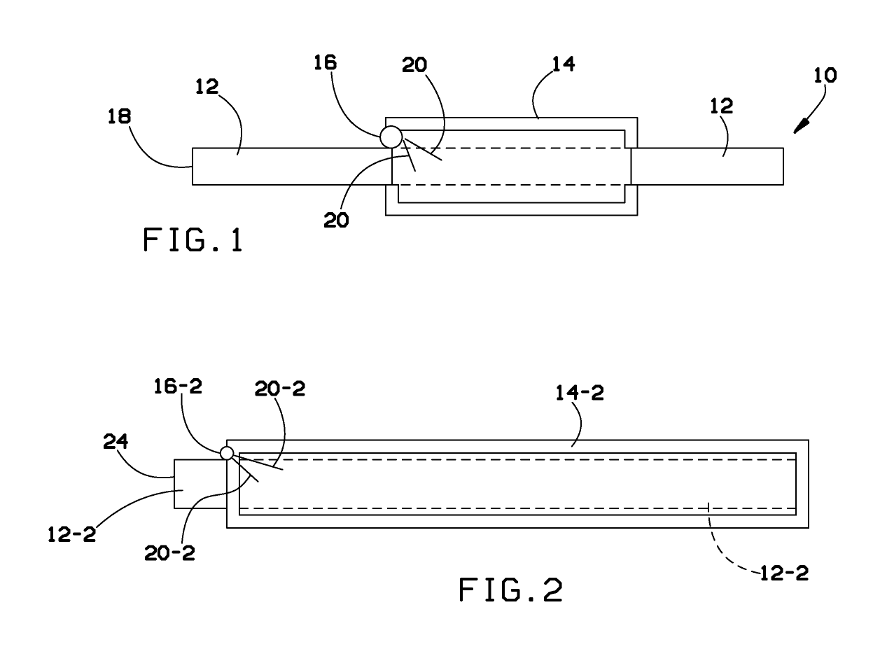

Referring to FIG. 1, an electrocautery tip 10 may include a cautery element 12. The cautery element 12 may be at least partially housed in an insulating housing 14. In one embodiment, the insulating housing 14 may be plastic. The electrocautery unit 10 may include a light 16. The light may be mounted to direct light towards an end 18 of the electrocautery tip 10. The light 16 may be, for example, an LED light. In the embodiment shown in FIG. 1, an electrical connection 20 may electrically connect the light with the same power supply used to energize the electrocautery unit. In this embodiment, the light 16 may illuminate when power is supplied to the electrocautery unit.

Referring to FIG. 2, an electrocautery unit 10-2, similar to electrocautery tip 10, may include a cautery element 12-2, an insulating housing 14-2, a light 16-2 and an electrical connection 20-2. The electrocautery unit 10-2 may include a suction opening 24 for suction cautery.

While the embodiments of FIGS. 1 and 2 describe certain electrocautery tools, embodiments of the present invention may be applied to any type of electrocautery units. For example, Valleylab, a division of Covidien (formerly Tyco Healthcare Group) provides various electrocautery units that may be designed to utilize a light to provide a tool according to embodiments of the present invention.

The light 16, 16-2 may be located near the tip of the electrocautery device. This location may provide light at or near the site chosen for electrocautery. The resulting electrocautery device, according to the present invention, may provide site illumination during electrocautery. Different lights, colors of lights, sizes of lights, and shape of lights may be varied as well as their position along the electrical circuit may be varied depending on the desired application of the electrocautery device.

Embodiments of the present invention may be used not only in the treatment of humans, but may also be used in the medical practices of veterinary medicine. Embodiments of the present invention may be used in external electrocautery as well as internal or laparoscopic electrocautery.

It should be understood, of course, that the foregoing relates to exemplary embodiments of the invention and that modifications may be made without departing from the spirit and scope of the invention as set forth in the following claims.

We claim:

1. An electrocautery device comprising:
an electrocautery tip;
a light source to supply light to at least the electrocautery tip.

2. The electrocautery device of claim 1, wherein the light source is formed in the electrocautery tip.

3. The electrocautery device of claim 1, wherein the light source illuminates when the user initiates electrocautery.

4. The electrocautery device of claim 1, wherein the light source illuminates when the electrocautery device is connected to a power supply, regardless of whether electrocautery is initiated.

5. The electrocautery device of claim 1, further comprising a switch, the switch turning on and off power to the light source.

6. The electrocautery device of claim 1, wherein the light source is at least one light emitting diode.

7. The electrocautery device of claim 1, wherein the electrocautery tip includes a suction opening.

8. The electrocautery device of claim 1, wherein the light source is focused to a user selected angle, size, and shape.

9. An electrocautery device comprising:
an electrocautery tip; and
a light emitting diode adapted to supply light to at least the electrocautery tip, wherein the light emitting diode is molded into the electrocautery tip.

10. The electrocautery device of claim 9, wherein the electrocautery tip is a suction electrocautery tip.
